(19) United States
(12) Patent Application Publication
(10) Pub. No.: US 2004/0223932 A1
(43) Pub. Date: Nov. 11, 2004

(54) Adhesive treatment for acne

(75) Inventors: Daniel L. Hedgpeth, Raleigh, NC (US); Upvan Narang, Raleigh, NC (US)

Correspondence Address: OLIFF & BERRIDGE, PLC, P.O. BOX 19928, ALEXANDRIA, VA, 22320, US

(73) Assignee: Closure Medical Corporation, Raleigh, NC

(21) Appl. No.: 10/428,984

(22) Filed: May 5, 2003

(51) Int. Cl. A61K 7/06; A61K 7/11

(52) U.S. Cl. 424/70.11

(57) ABSTRACT

A method of treating acne includes applying a polymerizable monomer adhesive composition to an area of skin afflicted with acne, optionally with at least one of an additional anti-acne agent or other active agent, and allowing the polymerizable monomer composition to polymerize to form a polymer film over the area of skin.

### BACKGROUND OF THE INVENTION

1. Field of Invention

The present invention relates to treatment and/or amelioration of symptoms of acne. More particularly, the present invention relates to methods and compositions for the treatment and/or amelioration of symptoms of acne.

2. Description of Related Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and polymers, they have found wide use due to the speed with which they cure, the strength of the resulting bond formed, and their relative ease of use. These characteristics have made the α-cyanoacrylate adhesives the primary choice for numerous applications such as bonding plastics, rubbers, glass, metals, wood, and, more recently, biological tissues.

It is known that monomeric forms of α-cyanoacrylates are extremely reactive, polymerizing rapidly in the presence of even minute amounts of an initiator, including moisture present in the air or on moist surfaces such as animal (including human) tissue. Monomers of α-cyanoacrylates are anionically polymerizable or free radical polymerizable, or polymerizable by zwitterions or ion pairs to form polymers. Once polymerization has been initiated, the cure rate can be very rapid.

Medical applications of 1,1-disubstituted ethylene adhesive compositions include use as an alternate or an adjunct to surgical sutures and/or staples in wound closure, as well as for covering and protecting surface wounds such as lacerations, abrasions, burns, stomatitis, sores, minor cuts and scrapes, and other wounds. When an adhesive is applied to surfaces to be joined, it is usually applied in its monomeric form, and the resultant polymerization gives rise to the desired adhesive bond.

U.S. Pat. Nos. 5,514,371, 5,514,372, 5,575,997, 5,624,669, and 5,582,834 to Leung et al. disclose the addition of a therapeutic agent in a cyanoacrylate composition. The cyanoacrylate adhesive forms a matrix for the therapeutic agent, with the therapeutic agent being released in vivo over time from the matrix during biodegradation of the polymer.

U.S. Pat. No. 5,762,955 to Smith discloses a treatment for healthy, damaged, diseased, or infected biological tissue by applying a bioadhesive coating in conjunction with a medication. The treatment is directed, in part, to treating external biological tissue that may be affected by harmful afflictions such as bruises, burns, dermatological afflictions, infections, gashes, wounds, herpes sores, canker sores, or intra-oral lesions, and skin cancers such as leukemia. Smith further discloses several medications that may be used including corticosteroids, fluoroouracil, obtundants, anesthetics, antibiotics, fungicides, anti-inflammatory agents, antibacterial agents, antiseptic agents, and other medications or combinations of medications used in processes for healing tissue, promoting or preventing blood clotting, destroying cancer cells, palliative treatments and killing of bacteria or viruses.

U.S. Pat. No. 4,880,416 to Horiuchi et al., discloses a dermal bandage of a pre-formed film-like adhesive material for preventing dermally applied ointments, creams, solutions, powders, etc. from falling off, and for delivering drugs, such as anti-fungal agents, to affected parts of the skin. U.S. Pat. Nos. 5,716,607 and 5,716,608, both to Byram et al., disclose the use of cyanoacrylate adhesives to prevent ionization radiation damage to skin. Such damage is prevented by applying the cyanoacrylate polymer to the skin to be protected. U.S. Pat. No. 5,653,769 to Barley, Jr., et al., discloses protecting skin areas from irritation due to contact with artificial devices such as prosthetics, bandages and casts by applying a cyanoacrylate polymer to the desired skin areas that otherwise would be prone to ulceration or irritation by the devices.

U.S. Pat. No. 4,287,177 to Nakashima et al. discloses a protective covering material for forming a film or coat on the skin or wound surface, wherein the film may contain an anti-fungal agent that is controllably released when the composition is placed in contact with the skin. U.S. Pat. Nos. 5,684,042; 5,753,699; 5,762,919; 5,783,177; and 5,811,091 to Greff et al. disclose a cyanoacrylate composition with a compatible anti-fungal agent to form an anti-fungal polymeric cyanoacrylate film to be applied on mammalian skin as wound dressings, wound bandages, surgical incise drapes, wound closure materials and the like.

Acne, medically defined as an inflammatory, follicular, papular, and pustular eruption involving the pilo sebaceous apparatus, is a condition that affects many individuals during their lifetime, and particularly during puberty. Simplex acne, also known as acne vulgaris, is a very prevalent and the most well-known form of acne, which takes the form of an eruption, predominantly of the face, upper back and chest, composed of comedones, cysts, papules, and pustules on an inflammatory base. The condition occurs in a majority of people during puberty, due to androgenic stimulation of sebum secretion, with plugging of follicles by keratinization, associated with proliferation of Propionibacterium acnes. However, other varieties of acne have been diagnosed and described in the literature. Unless otherwise noted herein, “acne” is used to generically refer to all such acne types.

In a less technical sense, acne generally includes the presence of inflamed areas of the skin, characterized by the presence of a pustule over the inflamed area. In minor cases, acne presents an aesthetic abnormality, but in major cases acne can present cysts in addition to the pustules, which can rupture and scar. In most cases, social pressures create a desire for acne to be treated, and visible symptoms to be minimized.

Physicians commonly prescribe medications in the form of powders, aerosols, liquids or creams for the treatment of acne. Such medications are even more commonly obtained by individuals “over-the-counter” for treatment of the same skin condition. Furthermore, although generally not contagious, it is possible that a higher concentration or proliferation of Propionibacterium acnes may be considered by many, rightly or wrongly, to be a possible communicative health risk.

Often the condition occurs on the face, chest and back areas of the body. Such areas are often exposed to higher levels of moisture (such as by perspiration) and/or come into frequent frictional contact with an individual's clothing. As a result of this moisture and/or frictional contact with clothing and the like, topically applied medications can be more easily removed from the affected areas of a patient's skin. Moreover, the topically applied medications are more prone to the effects of moisture that is present at the affected area of skin. These difficulties mean that treatment is significantly reduced, because the medication is not held in place for a sufficient time. As a result, the efficacy of the treatment is significantly reduced, and patients must frequently reapply medications so that the affected area receives proper treatment. Furthermore, the inconvenience of bandages due to constant or frequent movement and flexing of the skin, the small surface area usually involved, friction and moisture, make bandages impractical for minimizing frictional contact that occurs at the affected areas of a patient's skin, as well as impractical as a means to hold the medication in place for longer periods of time.

A still further concern, particularly in individuals during puberty, is the social aspect of treatment of acne. Social pressures are particularly acute at this age, and of utmost concern to many individuals. Accordingly, the visible presence of acne or acne treatments are highly disfavored. Such individuals thus prefer treatment protocols that are fast and minimally visible.

Accordingly, conventional treatment protocols for acne typically involve topical application of lotions and creams, which may optionally be medicated. A problem with such protocols, as described above, is that the lotion or cream may not remain in place for a sufficient length of time to provide effective treatment. An improved protocol involves application of a medicament, along with a bandage-like device to keep the medicament in place. For example, the ON-THE-SPOT® Acne Patch product, marketed by Neutrogena Corp., is a product that includes small (½ in. and ⅝ in. diameter) bandage-like patches that contain an acne-fighting ingredient. The patches are designed to be applied overnight and removed in the morning, as the patches are more aesthetically conspicuous than the acne itself.

Despite the known use of adhesives, such as described above, such adhesives have not been used in the treatment of acne. Instead, the majority of acne treatments currently on the market include topically applied medications that have the problems of being easily removed from the application site, and being prone to moisture effects. Therefore, there is a need for an alternative acne treatment that remains at the application site for longer periods, that provides fast, effective relief from the symptoms of acne, and that can be either easily removed or maintained in place for longer periods of time without being conspicuously present.

### SUMMARY OF THE INVENTION

The present invention provides methods for treating and/or ameliorating the symptoms of acne by applying a monomeric adhesive composition to the affected area. The monomeric adhesive composition of the present invention preferably comprises a polymerizable 1,1-disubstituted ethylene monomer such as a cyanoacrylate monomer, that may optionally include or be accompanied by an additional anti-acne agent. The composition keeps any active ingredients in contact with the affected skin area for a longer time, provides fast and effective treatment of acne, reduces or eliminates symptoms of acne, such as minor pain and itching sensations and inflammation, provides a healing environment, and, reduces infection of the affected areas.

The present invention provides an unexpected treatment composition and method for acne, because polymerizable monomers such as 1,1-disubstituted ethylene monomers and cyanoacrylates have not previously been used to treat or prevent such skin conditions. Moreover, while such polymerizable monomers have been variously used on other parts of the body, such as for sealing open wounds, their use for treating or preventing acne, on areas of skin such as the face, chest and back, is an entirely new and unexpected use of the materials.

The present treatment for acne is advantageous for several reasons. First, because acne affects areas of skin that are prone to moisture or frictional contact, it is often very difficult for the patient to keep a treatment agent in contact with the affected skin area for an extended period of time. This is either because frictional contact with the affected area tends to disturb any applied active agents, or because higher levels of moisture present in the area tend to affect or likewise disturb the applied agent. In addition, because many treatment products and methods are aesthetically non-preferred, especially when used on facial areas, the products are not used to the fullest extent possible to maximize their beneficial effect. The present invention addresses these drawbacks of the prior art, by providing a treatment method and composition that permit improved treatment of acne.

Because cyanoacrylates tend to polymerize rapidly to form a relatively robust polymerized film, cyanoacrylates applied to an affected area of a patient's skin can provide fast, effective protection over the affected area and other skin surfaces. By protecting the affected area from moisture and frictional contact, the cyanoacrylate compound can hold any applied active agents in place for a longer period of time and can accordingly significantly increase the time of exposure of an applied medication to the affected area, and ensure more effective treatment.

In addition to forming a stronger barrier to keep moisture and friction away from the affected area while maintaining active agents in contact with the area, cyanoacrylate compounds are also desirable for the treatment of acne because of their inherent anti-microbial properties. It has been demonstrated in at least some laboratory tests that some cyanoacrylate compounds or formulations provide microbial barrier and anti-microbial properties. Because of this anti-microbial property, such cyanoacrylate compounds and formulations may be especially desirable for treating acne, even without the introduction or pre-application of other anti-microbial or anti-acne agents. Moreover, the anti-microbial properties of such cyanoacrylate compounds and formulations may also be beneficial in instances where an anti-microbial or anti-acne agent is applied before, or together with, the cyanoacrylate, because the cyanoacrylate can continue to provide its anti-microbial effect even after the applied anti-microbial agent has been completely absorbed or used up.

Cyanoacrylate compounds are also useful carriers or delivery agents for anti-acne agents or other active agents. In this capacity, the cyanoacrylate compound provides the same protective, active and anti-acne benefits, but also offers the added benefit of enhancing the treatment of the affected area by controllably releasing the agent to the affected area.

### DETAILED DESCRIPTION OF PREFERRED EMBODIMENTS

This invention is directed to methods of and compositions for treating acne. In its method aspects, the present invention is directed to methods comprising applying a monomeric adhesive composition, with or without optional additives, to an afflicted area of skin. In its composition aspects, the present invention is directed to compositions comprising a monomeric adhesive composition and one or more anti-acne additives, which composition is useful in the treatment and/or amelioration of symptoms of acne.

According to embodiments of the present invention, the adhesive composition can be applied alone, or it can be applied subsequent to or concurrent with the application of a separate anti-acne or other active agent, such as an astringent. Furthermore, in embodiments, the monomeric adhesive composition can itself include an anti-acne or other active agent in addition to polymerizable monomer.

According to the present invention, “treat” (or other forms of the word such as “treating” and “treatment”) refers to employment of the methods and/or compositions against an established or progressing acne condition. “Treat” thus encompasses both amelioration of effects of acne, such as by reducing swelling, inflammation, irritation, itching, pain and the like, and active reduction of acne such as by speeding or assisting healing and reducing the microbial load that may be present in the afflicted area. Treatment is thus distinguished from prevention, which involves unaffected areas of skin in the absence of acne.

In embodiments of the invention, an adhesive composition can be used alone for treating acne. In such embodiments, a patient or care-provider simply applies the composition to an area of the skin afflicted with acne. The composition then is permitted to polymerize to form a robust polymeric coating that effectively covers and protects the affected or applied area of the patient's skin. Anti-acne properties of the composition may inhibit or kill microbes, and the robust polymeric coating that the composition forms protects the area from frictional contact with clothing or other skin surfaces so that prolonged treatment or prevention can be achieved.

Preferably, the composition is applied in a sufficient amount to entirely cover the desired area, which generally would correspond to an affected area where acne is present. In embodiments, the composition covers an additional area around the desired area.

According to the present invention, the adhesive composition is preferably permitted to substantially or fully polymerize to form a polymer film before the treated area is permitted to contact other surfaces, such as articles of clothing. Thus, for example, when the composition is applied to areas of skin, any adjoining skin surfaces are preferably kept separated from each other until the composition has substantially or fully polymerized, to prevent bonding the skin areas together. Likewise, for example where the composition is applied to areas of skin that lie beneath clothing, such as on the chest or back, the skin surfaces are preferably kept separated from the clothing until the composition has substantially or fully polymerized, to prevent bonding the skin area and clothing together.

Once applied and set, the polymerized composition is preferably retained on the application site for a time sufficient to permit treatment and/or amelioration of symptoms of acne. Thus, for example, the polymerized composition is preferably retained on the application site for a period of at least overnight, such as for from about 6 or about 8 to about 9 or about 14 hours, or any time in between. If desired, the polymerized composition is preferably retained on the application site for longer periods to permit increased treatment, such as for periods of at least 10 or at least 12 or at least 14 hours, and up to about 24, about 48 or about 72 hours. Of course, the polymerized composition can be retained on the application site for shorter or longer times, as desired.

In embodiments, the polymerized composition is retained on the application site for a given period of time, and is prematurely removed rather than allowing the polymerized composition to slough off of the application site. Thus, for example, although the polymerized composition would typically adhere to the application site on skin for about 5 to about 7 days before naturally sloughing off, it is preferred in embodiments that the polymerized composition be removed prior to such natural sloughing off. Such removal can be accomplished, for example, by removing the polymerized composition by using such measures as oil-based cleansers and/or mechanical rubbing, such as with a wash cloth. These embodiments provide the multiple benefits of treating acne using the polymerizable compositions, and cleansing the skin areas by way of the removal process and thus promoting personal hygiene and pore cleansing, while still providing removal of the formulation (or allowing the formulation to remain in place for periods of time but in a non-obtrusive manner) to avoid undesired stigma in social settings.

In terms of removal of the polymerized formulation, suitable biocompatible remover compositions are disclosed in, for example, U.S. patent application Ser. No. 09/962,268, filed Sep. 26, 2001, the entire disclosure of which is incorporated herein by reference. Alternatively, it is possible to formulate the polymerizable monomer composition itself to permit easy removal using oil-based cleansers, such as mineral oil or baby oil, and/or mechanical rubbing, such as with a wash cloth.

According to the present invention, the composition and method can be practiced to treat acne in any of its various stages. Thus, for example, the methods and compositions of the present invention can be used to treat acne in either its fully developed form, i.e., where fully developed comedones, cysts, papules, and/or pustules are present on an inflammatory base, or in its lesser (i.e., not fully) developed stages, i.e., where the inflammatory base, with or without initial formation of comedones, cysts, papules, or pustules, is beginning to form.

In further embodiments of the present invention, the adhesive composition can be applied over a medicament for treating acne. The medicament in this embodiment is not particularly limited, and can include any of the available medicaments or agents for the treatment of acne. The medicament can also be, or comprise, any suitable anti-acne agent, anti-microbial agent, anti-inflammatory or anesthetic, other active agents, or other treatment agents for acne, as described below. All such agents are generally referred to herein as “active agents” unless otherwise specified. In this embodiment, the medicament can be first applied to the affected or desired area, followed by application of a polymerizable adhesive composition. The medicament can be in any suitable form, including liquid, solid, powder, cream or the like, and can include only a medicament or can include other suitable additives such as diluents, carriers or the like. Where the medicament is in a liquid or a semi-liquid form, it is preferred that the medicament be permitted to dry, substantially or completely, prior to application of the adhesive composition. However, the adhesive composition can also be immediately applied over the applied medicament, or can be applied prior to drying of the medicament, if desired.

In embodiments of the present invention, an appropriate, preferably monomer-compatible, anti-acne or other active agent can be mixed with the polymerizable adhesive composition and a resultant composition applied to the affected or desired area. In this embodiment, the anti-acne or other active agent can be mixed with the polymerizable adhesive composition during manufacture (i.e. prior to packaging the materials), or immediately prior to use. However, the present invention is not limited to such embodiments. Thus, for example, the agent need not be monomer-compatible. In these embodiments, the agent can be mixed or combined with the polymerizable adhesive composition, usually just prior to application, and a resultant composition applied to the affected or desired area.

In further embodiments of the present invention the anti-acne or other active agent may also serve as a polymerization initiator or a stabilizer. Thus, the agent can provide not only a biological activity, but a chemical one as well.

Anti-acne or other active agents that also serve as polymerization initiators can initiate and/or accelerate the polymerization of the composition when applied to an affected or desired area of skin. Accelerated polymerization reduces the waiting time necessary after application, and makes the composition more convenient to apply. Suitable agents that can also serve as initiators include, but are not limited to, certain acidic and quaternary ammonium compounds. In embodiments where the agent also acts as a polymerization initiator or rate modifier, the present invention provides the additional advantage of not requiring that a further, separate polymerization initiator or rate modifier be used. Furthermore, in these embodiments, the agent is preferably located in a non-contacting relationship with the adhesive composition prior to use, so that premature polymerization of the adhesive composition does not occur.

Anti-acne or other active agents that also serve as stabilizers can extend the useful life of the composition. By increasing the useful life of the composition, the composition can be stored and packaged for longer periods of time without the risk of premature polymerization. Suitable agents that can also serve as stabilizers can include, but are not limited to, certain acidic and phenolic compounds. In embodiments where the agent also acts as a stabilizer for the adhesive composition, the present invention provides the additional advantage of not requiring that a further, separate stabilizer be used. Furthermore, in these embodiments, the agent is preferably located in a contacting relationship with the adhesive composition, such as being mixed with the adhesive composition, prior to use.

When the additives, such as an anti-acne or other active agent, are mixed with the monomer composition during storage, it is preferred that the mixture exhibit a sufficiently long shelf-life to permit economical commercial distribution of the mixture. Thus, for example, the mixture should exhibit a shelf-life, as measured at room temperature and moderate humidity (about 60% relative humidity), of at least about one year, and preferably at least about two or even at least about three years. Where the additive and monomer are not mixed during storage, it is still preferred that the separate components exhibit similar shelf-lives to those of a mixed composition. As used herein, “shelf-life” refers to the amount of time the container and composition therein can be held at approximately room temperature (21-25° C.) without degradation of the composition and/or container occurring to the extent that the composition and container cannot be used in the manner and for the purpose for which they were intended. Thus, while some degradation to either or both of the composition and container can occur, it must not be to such an extent that the composition and/or container is no longer useable. Shelf-life can thus be limited by physical or aesthetic changes to the containers or products contained therein, by chemical reactions occurring within the composition being stored, by chemical reactions between the container and the composition being stored, by degradation of the container itself, and the like.

Although a mixture of anti-acne or other active agent and polymerizable monomer, according to the present invention, is not limited to a specific ratio of agent to polymerizable monomer, the agent is preferably present in an effective amount, preferably a therapeutically effective amount for treating acne.

When mixed or combined immediately prior to use, the anti-acne or other active agent can be mixed with the polymerizable monomer composition in a suitable container and thereafter applied. Alternatively, mixing can be conducted during the application process, for example by using an applicator that is loaded with the agent, which thereby mixes the agent with the adhesive composition during application.

Suitable anti-acne agents include, but are not limited to, known agents such as resorcinol acetate, resorcinol, salicylic acid, benzyl alcohol, sodium peroxide, organic peroxides such as benzoyl peroxide, alpha hydroxy acids and detergents. Still further suitable anti-acne agents include, but are not limited to, known agents such as acetyltannic acid, alkanet, alkannin, aluminum acetate solution, aluminum acetotartrate, aluminum ammonium sulfate, aluminum chlorate, aluminum chloride, aluminum hydroxychloride, aluminum beta-naphtholdisulfonate, aluminum potassium sulfate, aluminum sodium sulfate, aluminum subacetate solution, ammonium ferric sulfate, baicalein, bayberry bark, bismuth oxide, bismuth subgallate, bismuth tannate, boric acid, calamine, calcium hydroxide, catechu black, chromium trioxide, cimicifuga, condurangin, cormus, cupric citrate, dichloroacetic acid, eucalyptus gum, ferric chloride, ferric subsulfate solution, ferrous sulfate, formic acid, gallic acid, gambir, geranium, germanium, hamamelis, iodic acid, kino, lead acetate, matico, methionic acid, myrrh, nutgall, potassium permanganate, quercus, rumex, silver bromide, silver lactate, silver nitrate, sodium borate, sodium formate, starch, tannic acid, tannoform, trillium, zinc acetate, zinc carbonate, zinc chloride, zinc iodide, zinc oxide, zinc permanganate, zinc peroxide, zinc p-phenolsulfonate, zinc salicylate, zinc stearate, zinc sulfate, zinc tannate, mixtures thereof, and the like.

Benzoyl peroxide is a colorless, odorless, tasteless crystalline solid that is stable at ordinary room temperatures. It is also a strong oxidizing agent that may be used as an antibacterial and keratolytic agent in the treatment of acne. Finely divided benzoyl peroxide often is incorporated in a conventional cream or ointment for convenience in applying it to the skin. However, because of the powerful oxidizing properties of benzoyl peroxide, the inclusion of it in conventional ointment or cream bases often results in unstable compositions that display an unacceptably rapid loss in keratolytic potency. Benzoyl peroxide has been reported to be irritating to skin when applied at concentrations appropriate for the treatment of acne. The same is also generally true of conventional detergent-based anti-acne compositions. For example, detergent compositions based upon salts of lauryl sulfates (e.g., ammonium lauryl sulfate) are known to cause skin irritation. Consequently, conventional anti-acne compositions containing benzoyl peroxide and/or detergents often contain one or more moisturizers in order to minimize skin irritation associated with the anti-acne agent. The same can be applied to the present invention. Thus, where benzoyl peroxide and/or detergents are used as the anti-acne agent, the composition may further include one or more moisturizers, as appropriate.

Organic peroxides that may be included in the compositions of the present invention include any pharmaceutically acceptable organic peroxide, such as, for example, benzoyl peroxide, lauroyl peroxide, and carbamide peroxide. Preferably, the organic peroxide is benzoyl peroxide. The amount of organic peroxide present in the compositions of the invention may be from about 1 weight percent to about 20 weight percent, based upon the weight of the composition. Preferably, the organic peroxide is present in an amount from about 2.5 weight percent to about 10 weight percent.

Alpha hydroxy acids that may be included in the topical compositions of the present invention include any pharmaceutically acceptable alpha hydroxy acid, such as, for example, glycolic acid, lactic acid, 2-hydroxydecanoic acid, 2-hydroxystearic acid and malic acid. Preferably, the alpha hydroxy acid is one that is commonly used in topical compositions for treating acne, such as glycolic acid or lactic acid. Most preferably, the alpha hydroxy acid is glycolic acid. The amount of alpha hydroxy acid present in the compositions of the invention may be from about 0.1 weight percent to about 15 weight percent, based upon the weight of the composition. Preferably, the alpha hydroxy acid is present in an amount from about 1 weight percent to about 10 weight percent.

Detergents that may be included in the compositions of the present invention include any pharmaceutically acceptable detergent. Such detergents include, for example, sodium potassium lauryl sulfate, cocamidopropyl betaine, sodium cocoylisethionate, and disodium cocoamphopropionate. Preferably, the detergent is sodium potassium lauryl sulfate or cocamidopropyl betaine. The amount of detergent present in the compositions of the invention may be from about 15 weight percent to about 60 weight percent, based upon the weight of the composition. Preferably, the detergent is present in an amount from about 25 weight percent to about 40 weight percent.

Suitable anti-microbial agents include, but are not limited to, known agents such as parabens, cresols, azoles, allylamines, pollyenes, acidics, mercurials, quaternary ammonium compounds, other agents, non-polymer-stabilized compounds, i.e., that are not complexed with or otherwise part of a polymer species, mixtures thereof, and the like. Such anti-microbial agents should preferably be present in a therapeutically effective amount, particularly in cases where higher amounts may otherwise be toxic to the patient. Suitable such agents are disclosed in, for example, U.S. patent application Ser. No. 09/898,092, filed Jul. 5, 2001, the entire disclosure of which is incorporated herein by reference, and are designated therein as “anti-fungal agents.”.

In addition, in embodiments where monomer additives including, but not limited to those listed above, are insoluble with the monomer composition and/or that would cause premature polymerization of the monomer, the additive can be applied to a skin area before applying the monomer composition. In such embodiments, the additive and the monomer composition can be provided, for example, in separate packages in a kit.

In other embodiments, where such additives are soluble with the monomer composition and/or would not cause premature polymerization of the monomer, the additives can be combined with the monomer composition during manufacture of the composition. Moreover, in cases where the additive is soluble with the monomer composition, the additive can be applied before the monomer composition is applied, it can be pre-mixed with and applied together with the monomer composition, it can be mixed together with the monomer composition immediately before application, or it can even be applied after the monomer composition has been applied. As a result, in cases where a soluble additive is to be applied, the additive and the composition can be provided in a kit where the additive and the monomer composition are pre-mixed, or the additive and the monomer composition can be provided separately to be applied separately or mixed together immediately prior to, during, or after application.

Although a mixture of additive and monomer composition according to the present invention is not limited to a specific ratio of additive to polymerizable monomer, the additive is preferably present in an effective amount, and preferably in a therapeutically effective amount.

When mixed immediately prior to use, the additive can be mixed with the polymerizable monomer composition in a suitable container and thereafter applied. Alternatively, mixing can be conducted during the application process, for example by using an applicator loaded with the additive, which thereby mixes the additive with the adhesive composition during application.

In addition, as discussed above with respect to suitable anti-acne and other active agents, various skin care additives may also serve as polymerization initiators or rate modifiers. Also, suitable skin care additives may serve as stabilizers for the adhesive composition.

In embodiments, the monomer composition and/or its packaging can be sterilized. However, sterilization is by no means required, particularly in view of the fact that most commercially available products for the treatment of acne are not sterilized. Furthermore, whether or not the composition and container is sterilized, the composition can further include one or more suitable preservative, as described below.

Sterilization of the monomer composition and/or its packaging can be accomplished by techniques known to the skilled artisan, and is preferably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. A preferred method is electron beam irradiation, as described in U.S. Pat. No. 6,143,805, the entire disclosure of which is incorporated herein by reference. The composition should also show low levels of toxicity to living tissue during its useful life. In preferred embodiments of the present invention, the composition is sterilized to provide a Sterility Assurance Level (SAL) of at least 10⁻³. In embodiments, the Sterility Assurance Level may be at least 10⁻⁴, or may be at least 10⁻⁵, or may be at least 10⁻⁶.

The monomer (including prepolymeric) adhesive composition may include one or more polymerizable monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687, 5,928,611 and 6,183,593, U.S. patent application Ser. No. 09/430,177, filed on Oct. 29, 1999, and U.S. Pat. No. 6,183,593, which are hereby incorporated in their entirety by reference herein.

Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

Preferred α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Other suitable cyanoacrylates for use in the present invention also include, but are not limited to, alkyl ester cyanoacrylate monomers such as those having the formula

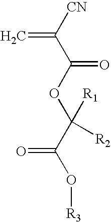

wherein R₁ and R₂ are, independently H, a straight, branched or cyclic alkyl, or are combined together in a cyclic alkyl group, and R₃ is a straight, branched or cyclic alkyl group. Preferably, R₁ is H or a C₁, C₂ or C₃ alkyl group, such as methyl or ethyl; R₂ is H or a C₁, C₂ or C₃ alkyl group, such as methyl or ethyl; and R₃ is a C₁-C₁₆ alkyl group, more preferably a C₁-C₁₀ alkyl group, such as methyl, ethyl, propyl, butyl, pentyl, hexyl, heptyl, octyl, nonyl or decyl, and even more preferably a C₂, C₃ or C₄ alkyl group. Such alkyl ester cyanoacrylates and other suitable monomers are disclosed in, for example, U.S. patent applications Ser. No. 09/630,437, filed Aug. 2, 2000, and Ser. No. 09/919,877, filed Aug. 2, 2001, the entire disclosures of which are incorporated herein by reference.

Examples of preferred alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA). BLCA may be represented by the above formula, wherein R₁ is H, R₂ is methyl and R₃ is butyl. BGCA may be represented by the above formula, wherein R₁ is H, R₂ is H and R₃ is butyl. ELCA may be represented by the above formula, wherein R₁ is H, R₂ is methyl and R₃ is ethyl. EGCA may be represented by the above formula, wherein R₁ is H, R₂ is H and R₃ is ethyl.

The composition may optionally also include at least one other plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to silica particles, tributyl citrate, acetyl tri-n-butyl citrate (ATBC), polymethylmethacrylate, silicone oils, siloxanes, and others as listed in U.S. Pat. No. 6,183,593, the disclosure of which is incorporated in its entirety by reference herein. Specific examples of the silicone oils and siloxanes include, for example, but are not limited to, polydimethylsiloxane, hexadimethylsilazane.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. Pat. No. 6,310,166, the disclosures of which are hereby incorporated in their entireties by reference herein.

The composition may optionally also include thickeners. Suitable thickeners may include poly (2-ethylhexy methacrylate), poly(2-ethylhexyl acrylate) and others as listed in U.S. Pat. No. 6,183,593, the disclosure of which is incorporated by reference herein in its entirety.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties by reference herein.

The composition may optionally also include one or more stabilizers, preferably both at least one anionic vapor phase stabilizer and at least one anionic liquid phase stabilizer. These stabilizing agents may inhibit premature polymerization. Suitable stabilizers may include those listed in U.S. Pat. No. 6,183,593, the disclosure of which is incorporated by reference herein in its entirety. Furthermore, certain stabilizers may also function as active agents, such as, for example, various acidic anti-microbials, as identified above.

The stability, and thus the shelf-life, of some monomeric adhesive compositions can be further enhanced and extended through careful regulation of the packaging. Treated (e.g., fluorinated polymer) packaging such as that disclosed in copending U.S. patent application Ser. No. 09/430,289, filed Oct. 29, 1999, which is hereby incorporated by reference herein in its entirety, is preferred and may reduce the amount of stabilizer that is combined into the composition. As mentioned above, certain stabilizers including, but not limited to, certain acidics can also function as active agents. In this case, the amount of the active agent/stabilizer material is either not reduced below a level to provide the desired effect, or a further active agent/non-stabilizing agent is added to ensure that the desired effect is provided.

The compositions may also include pH modifiers to control the rate of degradation of the resulting polymer, as disclosed in U.S. Pat. No. 6,143,352, the entire disclosure of which is hereby incorporated by reference herein in its entirety.

Compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites, etc. Additional examples of formaldehyde scavenger compounds useful in this invention and methods for their implementation can be found in U.S. Pat. Nos. 5,328,687, 5,514,371, 5,514,372, 5,575,997, 5,582,834 and 5,624,669, all to Leung et al., which are hereby incorporated herein by reference in their entireties.

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain fibrous reinforcement and colorants such as dyes, pigments, and pigment dyes. Examples of suitable fibrous reinforcement include PGA microfibrils, collagen microfibrils, and others as described in U.S. Pat. No. 6,183,593, the disclosure of which is incorporated by reference herein in its entirety.

The polymerizable compositions useful in the present invention may also further contain one or more preservatives, for prolonging the storage life of the composition. Suitable preservatives, and methods for selecting them and incorporating them into adhesive compositions, are disclosed in U.S. patent application Ser. No. 09/430,180, the entire disclosure of which is incorporated herein by reference. Such preservatives can be in addition to any anti-microbial agent that may or may not be added to the composition, as described above. Such preservatives can be included irrespective of whether the composition and containers are sterilized.

In embodiments of the present invention, the composition and/or its applicator may contain materials such as a polymerization initiator, accelerator, rate-modifier, and/or cross-linking agent for initiating polymerization and/or cross-linking of the polymerizable monomer material. Suitable materials and applicators and packaging systems are disclosed in U.S. Pat. Nos. 5,928,611, 6,352,704 and 6,455,064 and U.S. patent application Ser. Nos. 09/430,177, 09/430,289, 09/430,290, and 09/430,180 filed Oct. 29, 1999; Ser. No. 09/385,030 filed Aug. 30, 1999; and Ser. No. 09/176,889 filed Oct. 22, 1998; the entire disclosures of which are incorporated herein by reference.

According to the present invention, any suitable applicator can be used to apply the composition to the affected areas of skin. Suitable applicators and packaging systems are disclosed in, for example, U.S. Pat. Nos. 5,928,611, 6,352,704 and 6,455,064 and U.S. patent application Ser. Nos. 09/430,177, 09/430,289, 09/430,290, and 09/430,180 filed Oct. 29, 1999; Ser. No. 09/385,030 filed Aug. 30, 1999; Ser. No. 09/176,889 filed Oct. 22, 1998, and Ser. No. 09/898,006 filed Jul. 5, 2001; the entire disclosures of which are incorporated herein by reference.

### EXAMPLES

A 2-octyl cyanoacrylate monomer composition is prepared by adding 2 wt % salicylic acid to 2-octyl cyanoacrylate monomer. The mixture is stirred.

The characteristics of the composition are observed at about one minute after preparation and later at least twenty-four hours after preparation. The results of the observations show that the solution remains clear, indicating that salicylic acid is soluble in the monomer and does not cause premature polymerization.

The composition is then applied to affected areas of skin showing the characteristics of various stages of acne. The monomer composition polymerizes in under one minute, resulting in a polymerized film of material covering the affected area. The polymerized film is left in place overnight (about nine hours), allowing the salicylic acid to provide an anti-acne effect. Although the polymerized film would naturally remain in place for at least three days, the film is removed using baby oil.

While the invention has been described with reference to preferred embodiments, the invention is not limited to the specific examples given, and other embodiments and modifications can be made by those skilled in the art without departing from the spirit and scope of the invention.

What is claimed is:

1. A method of treating acne, comprising:
a) applying an adhesive composition comprising a polymerizable monomer to an area of skin that is afflicted with acne; and
b) allowing said polymerizable monomer to polymerize to form a polymer film over said area of skin.

2. The method of claim 1, wherein said area of skin exhibits inflammation associated with acne, but does not exhibit a fully developed comedone, cyst, papule, or pustule.

3. The method of claim 1, wherein said area of skin exhibits both inflammation associated with acne and a fully developed comedone, cyst, papule, or pustule.

4. The method of claim 1, wherein said acne is simplex acne.

5. The method of claim 1, wherein said polymerizable monomer comprises a 1,1-disubstituted ethylene monomer.

6. The method of claim 1, wherein said polymerizable monomer is an α-cyanoacrylate monomer.

7. The method of claim 1, wherein said polymerizable monomer comprises at least one member selected from the group consisting of ethyl cyanoacrylate, butyl cyanoacrylate, and 2-octyl cyanoacrylate.

8. The method of claim 1, further comprising combining at least one agent selected from the group consisting of anti-acne agents, anti-microbial agents, anti-inflammatory agents, anesthetics, and skin care additives with the polymerizable monomer composition, wherein the at least one agent serves as a polymerization initiator for said polymerizable monomer composition.

9. The method of claim 1, wherein said composition further comprises at least one stabilizing agent for said polymerizable monomer.

10. The method of claim 9, wherein said stabilizing agent is also at least one of an anti-acne agent, an anti-microbial agent and a skin care additive.

11. The method of claim 1, wherein said composition comprises at least one plasticizer.

12. The method of claim 11, wherein the plasticizer comprises at least one member selected from the group consisting of silica particles, tributyl citrate, acetyl tributyl citrate, polymethylmethacrylate, silicone oils, and siloxanes.

13. The method of claim 1, wherein the composition further comprises at least one agent selected from the group consisting of an anti-acne agent, an anti-microbial agent, an anti-inflammatory agent, an anesthetic, and a skin care additive.

14. The method of claim 1, wherein the composition further comprises at least one anti-acne agent.

15. The method of claim 14, wherein said at least one anti-acne agent is selected from the group consisting of resorcinol acetate, resorcinol, salicylic acid, benzyl alcohol, sodium peroxide, organic peroxides, alpha hydroxy acids, and detergents.

16. The method of claim 14, wherein said at least one anti-acne agent is salicylic acid.

17. The method of claim 14, wherein said at least one anti-acne agent is an organic peroxide selected from the group consisting of benzoyl peroxide, lauroyl peroxide, and carbamide peroxide.

18. The method of claim 14, wherein said at least one anti-acne agent is an alpha-hydroxy acid selected from the group consisting of glycolic acid, lactic acid, 2-hydroxydecanoic acid, 2-hydroxystearic acid and malic acid.

19. The method of claim 14, wherein said at least one anti-acne agent is a detergent selected from the group consisting of sodium potassium lauryl sulfate, cocamidopropyl betaine, sodium cocoylisethionate, and disodium cocoamphopropionate.

20. The method of claim 14, wherein said at least one anti-acne agent is selected from the group consisting of acetyltannic acid, alkanet, alkannin, aluminum acetate solution, aluminum acetotartrate, aluminum ammonium sulfate, aluminum chlorate, aluminum chloride, aluminum hydroxychloride, aluminum beta-naphtholdisulfonate, aluminum potassium sulfate, aluminum sodium sulfate, aluminum subacetate solution, ammonium ferric sulfate, baicalein, bayberry bark, bismuth oxide, bismuth subgallate, bismuth tannate, boric acid, calamine, calcium hydroxide, catechu black, chromium trioxide, cimicifuga, condurangin, cormus, cupric citrate, dichloroacetic acid, eucalyptus gum, ferric chloride, ferric subsulfate solution, ferrous sulfate, formic acid, gallic acid, gambir, geranium, germanium, hamamelis, iodic acid, kino, lead acetate, matico, methionic acid, myrrh, nutgall, potassium pernanganate, quercus, rumex, silver bromide, silver lactate, silver nitrate, sodium borate, sodium formate, starch, tannic acid, tannoform, trillium, zinc acetate, zinc carbonate, zinc chloride, zinc iodide, zinc oxide, zinc permanganate, zinc peroxide, zinc p-phenolsulfonate, zinc salicylate, zinc stearate, zinc sulfate, and zinc tannate.

21. The method of claim 1, wherein the composition further comprises at least one anti-microbial agent selected from the group consisting of parabens, cresols, azoles, allylamines, pollyenes, acidics, mercurials, quaternary ammonium compounds, and non-polymer-stabilized compounds.

22. The method of claim 13, wherein the at least one agent is mixed with the polymerizable monomer composition immediately prior to applying the polymerizable monomer composition to the area of skin.

23. The method of claim 13, wherein the at least one agent is mixed with the polymerizable monomer composition during manufacture of the polymerizable monomer composition.

24. The method of claim 1, wherein said composition has a Sterility Assurance Level (SAL) of 10⁻³-10⁻⁶.

25. The method of claim 1, further comprising applying at least one agent selected from the group consisting of an anti-acne agent, an anti-microbial agent, an anti-inflammatory agent, an anesthetic, and a skin care additive to the area of skin before applying the adhesive composition.

26. The method of claim 25, further comprising allowing the at least one applied agent to substantially dry before applying the adhesive composition.

27. The method of claim 25, wherein the anti-acne agent is applied and is selected from the group consisting of resorcinol acetate, resorcinol, salicylic acid, benzyl alcohol, sodium peroxide, organic peroxides, alpha hydroxy acids, and detergents.

28. The method of claim 25, wherein the anti-acne agent is applied and is salicylic acid.

29. The method of claim 25, wherein the anti-acne agent is applied and is an organic peroxide selected from the group consisting of benzoyl peroxide, lauroyl peroxide, and carbamide peroxide.

30. The method of claim 25, wherein the anti-acne agent is applied and is an alpha-hydroxy acid selected from the group consisting of glycolic acid, lactic acid, 2-hydroxydecanoic acid, 2-hydroxystearic acid and malic acid.

31. The method of claim 25, wherein the anti-acne agent is applied and is a detergent selected from the group consisting of sodium potassium lauryl sulfate, cocamidopropyl betaine, sodium cocoylisethionate, and disodium cocoamphopropionate.

32. The method of claim 25, wherein the anti-microbial agent is applied and is selected from the group consisting of parabens, cresols, and non-polymer-stabilized compounds.

33. The method of claim 25, wherein the anti-acne agent is applied and is selected from the group consisting of acetyltannic acid, alkanet, alkannin, aluminum acetate solution, aluminum acetotartrate, aluminum ammonium sulfate, aluminum chlorate, aluminum chloride, aluminum hydroxychloride, aluminum beta-naphtholdisulfonate, aluminum potassium sulfate, aluminum sodium sulfate, aluminum subacetate solution, ammonium ferric sulfate, baicalein, bayberry bark, bismuth oxide, bismuth subgallate, bismuth tannate, boric acid, calamine, calcium hydroxide, catechu black, chromium trioxide, cimicifuga, condurangin, cormus, cupric citrate, dichloroacetic acid, eucalyptus gum, ferric chloride, ferric subsulfate solution, ferrous sulfate, formic acid, gallic acid, gambir, geranium, germanium, hamamelis, iodic acid, kino, lead acetate, matico, methionic acid, myrrh, nutgall, potassium permanganate, quercus, rumex, silver bromide, silver lactate, silver nitrate, sodium borate, sodium formate, starch, tannic acid, tannoform, trillium, zinc acetate, zinc carbonate, zinc chloride, zinc iodide, zinc oxide, zinc permanganate, zinc peroxide, zinc p-phenolsulfonate, zinc salicylate, zinc stearate, zinc sulfate, and zinc tannate.

34. The method of claim 1, wherein said adhesive composition is applied directly to said area of skin, and said adhesive composition does not include an anti-acne agent, an anti-microbial agent or a skin care additive.

35. The method of claim 34, wherein said polymer film has anti-microbial effects at said area of skin.

36 A method of treating acne, the method comprising the steps of:
a. applying at least one agent selected from the group consisting of an anti-acne agent, an anti-microbial agent, an anti-inflammatory agent, an anesthetic, and a skin care additive to an area of skin that is afflicted with acne;
b. applying a polymerizable monomer composition to said area of skin over the at least one applied agent, wherein said composition comprises a 1,1-disubstituted ethylene monomer; and
c. allowing said polymerizable monomer composition to polymerize to form a polymer film over said area of skin and said at least one agent.

37. A method of treating acne, the method comprising:
a. combining a polymerizable monomer composition and at least one agent selected from the group consisting of an anti-acne agent, an anti-microbial agent, an anti-inflammatory agent, an anesthetic, and a skin care additive to form a mixture;
b. applying said mixture to an area of skin that is afflicted with acne; and
c. allowing said mixture to polymerize to form a polymer film over said area of skin.

38. A composition, comprising:
a polymerizable 1,1-disubstituted ethylene monomer, and an anti-acne agent present in an effective amount to treat or ameliorate symptoms of acne.

39. The composition of claim 38, wherein said anti-acne agent is selected from the group consisting of resorcinol acetate, resorcinol, salicylic acid, benzyl alcohol, sodium peroxide, organic peroxides, alpha hydroxy acids, and detergents.

40. The composition of claim 38, wherein said anti-acne agent is salicylic acid.

41. The composition of claim 38, wherein said anti-acne agent is an organic peroxide selected from the group consisting of benzoyl peroxide, lauroyl peroxide, and carbamide peroxide.

42. The composition of claim 38, wherein said anti-acne agent is an alpha-hydroxy acid selected from the group consisting of glycolic acid, lactic acid, 2-hydroxydecanoic acid, 2-hydroxystearic acid and malic acid.

43. The composition of claim 38, wherein said anti-acne agent is a detergent selected from the group consisting of sodium potassium lauryl sulfate, cocamidopropyl betaine, sodium cocoylisethionate, and disodium cocoamphopropionate.

44. The composition of claim 38, wherein the anti-acne agent is selected from the group consisting of acetyltannic acid, alkanet, alkannin, aluminum acetate solution, aluminum acetotartrate, aluminum ammonium sulfate, aluminum chlorate, aluminum chloride, aluminum hydroxychloride, aluminum beta-naphtholdisulfonate, aluminum potassium sulfate, aluminum sodium sulfate, aluminum subacetate solution, ammonium ferric sulfate, baicalein, bayberry bark, bismuth oxide, bismuth subgallate, bismuth tannate, boric acid, calamine, calcium hydroxide, catechu black, chromium trioxide, cimicifuga, condurangin, cormus, cupric citrate, dichloroacetic acid, eucalyptus gum, ferric chloride, ferric subsulfate solution, ferrous sulfate, formic acid, gallic acid, gambir, geranium, germanium, hamamelis, iodic acid, kino, lead acetate, matico, methionic acid, myrrh, nutgall, potassium permanganate, quercus, rumex, silver bromide, silver lactate, silver nitrate, sodium borate, sodium formate, starch, tannic acid, tannoform, trillium, zinc acetate, zinc carbonate, zinc chloride, zinc iodide, zinc oxide, zinc permanganate, zinc peroxide, zinc p-phenolsulfonate, zinc salicylate, zinc stearate, zinc sulfate, and zinc tannate.

45. The composition of claim 38, wherein said anti-acne agent is soluble in said monomer and does not cause premature polymerization of the monomer.

46. The composition of claim 38, wherein said composition has a shelf-life of at least about two years.

47. The composition of claim 38, wherein said polymerizable monomer is an α-cyanoacrylate monomer.

48. The composition of claim 38, wherein said polymerizable monomer comprises at least one member selected from the group consisting of ethyl cyanoacrylate, butyl cyanoacrylate, and 2-octyl cyanoacrylate.
